(19) United States
(12) Patent Application Publication
(10) Pub. No.: US 2025/0122461 A1
(43) Pub. Date: Apr. 17, 2025

(54) Effective gravity and electro field control device to improve cell membrane permeability

(71) Applicant: LIIN TECHNOLOGIES INCORPORATED, Hampton, VA (US)

(72) Inventors: Hargsoon YOON, Yorktown, VA (US); Huu Vu PHAM, Virginia Beach, VA (US)

(21) Appl. No.: 18/379,227

(22) Filed: Oct. 12, 2023

(51) Int. Cl. C12M 1/42; C12M 1/34

(52) U.S. Cl. CPC C12M 35/02; C12M 35/04; C12M 41/40

(57) ABSTRACT

The present invention relates to an effective gravity and electric field control device that increases cell membrane permeability by implementing a zero-gravity, microgravity, low-gravity, or weighted gravity state in a module accommodating cells. In addition, the present invention relates to an effective gravity and electric field control device that combines a method for changing gravity and a method for applying an electric field.

The effective gravity and electric field control device according to one embodiment of the present invention includes an external module in which a movement space of an internal module is provided and of which at least one physical quantity related to movement is controlled and the internal module moving independently from the external module within the movement space by an external force transmitted through the external module, and the internal module includes an internal space where the target cell and a specific substance to be transfected into the target cell are loaded in a suspension state.

### FIELD OF THE INVENTION

The present invention disclosed herein relates to an effective gravity and electric field control device for increasing cell membrane permeability, and more specifically, to an effective gravity and electric field control device implementing a zero gravity, microgravity, low gravity, or weighted gravity state in a module accommodating cells.

### BACKGROUND ART

Gene therapy is a treatment that seeks to correct specific cells by transfecting genetic material such as DNA or RNA into cells. As a method for transfecting genetic material into cells, a method in which genes are wrapped with chemicals such as liposomes and then the genes are fused with cells and a method in which a therapeutic gene is inserted into a virus gene and then cells are infected are used.

In recent years, “electroporation” has been suggested as a method for inserting genetic material into cells. Electroporation is a physical method capable of using electric pulses to create a temporary hole in a cell membrane and through the hole, transfecting genetic material into a cell. Electroporation has the advantage of being able to process large quantities compared to methods using viruses or the like in the related art, but has been pointed out to have problems such as unintended cell destruction in a process of applying electric pulses.

Meanwhile, in general, zero gravity or microgravity is a state in which the Earth's gravity is canceled out and the effective gravity is brought close to 0 (zero), meaning a state in which there is no weight inside a satellite or spacecraft orbiting the Earth or a free-falling elevator. Weighted gravity is a state in which a gravitational load greater than the Earth's gravity is applied, which may be implemented through changes such as rapidly rotating an object.

For a method presented for applying microgravity to cells in the related art, there is a rotating wall vessel device for implementing microgravity using a rotation method and allowing experiments to be performed on cells floating in suspension. Some experimental devices that intentionally change the gravity applied to an object have been suggested, but most of the devices are made of complex and expensive equipment, which makes it difficult to easily implement a change in gravity.

### SUMMARY OF THE INVENTION

The present invention provides a device for applying a change in gravity to target cells and a method for increasing cell membrane permeability, as a method for increasing cell membrane permeability to transfect a specific substance into the target cells.

The present invention also provides a device for applying a zero gravity, microgravity, low gravity, or weighted gravity state to a module in which a target cell is accommodated to cause perforations in a cell membrane through shape deformation of the target cell.

In addition, the present invention provides a device and a method capable of improving limitations of experimental equipment in the related art and effectively increasing cell membrane permeability by allowing the combination of electroporation in the related art while controlling effective gravity to increase cell membrane permeability.

In addition, the present invention provides a device and a method capable of inserting a specific substance into a large number of target cells even in a short period of time and minimizing damage to the cells.

To solve the above-mentioned limitations, in accordance to an embodiment of the present invention, there is provided an effective gravity and electric field control device that implements a zero gravity, microgravity, low gravity, or weighted gravity state in a target cell, the effective gravity and electric field control device including an external module in which a movement space of an internal module is provided and of which at least one physical quantity related to movement is controlled, the internal module moving independently from the external module within the movement space by an external force transmitted through the external module, a position detection sensor configured to detect a position of the internal module within the movement space, a controller configured to generate a control signal for controlling at least one physical quantity related to the movement of the external module based on the position of the internal module detected by the position detection sensor, and an actuator configured to move the external module so that the external module has the at least one physical quantity according to the control signal, in which wherein the internal module includes an internal space where the target cell and a specific substance to be transfected into the target cell are loaded in a suspension state.

Further, in the effective gravity and electric field control device in accordance with an embodiment of the present invention, the controller may be configured to generate a first control signal for controlling the actuator so that an external force is applied to the internal module through the external module in a first section and generate a second control signal for controlling the actuator by referring to the position of the internal module after the external force applied to the internal module is released in a second section after the first section.

Further, in the effective gravity and electric field control device in accordance with an embodiment of the present invention, the first section may be an acceleration section and the second section may be a microgravity section.

Further, the effective gravity and electric field control device in accordance with an embodiment of the present invention may further include an electrode unit configured to apply current to the internal space, and the electrode unit may include a plurality of electrodes installed along a wall of the internal module.

Further, in the effective gravity and electric field control device in accordance with an embodiment of the present invention, the electrode unit may further include a current source configured to supply current to the plurality of electrodes and a multiplexer configured to selectively distribute signals applied from the current source to the plurality of electrodes, and the current source and the multiplexer may form an electric field and a current flow in various directions in the internal space through the plurality of electrodes.

Further, in the effective gravity and electric field control device in accordance with an embodiment of the present invention, the controller may be configured to generate an electric signal for forming an electric field in the internal space through the electrode unit in the first section or the second section.

Further, in the effective gravity and electric field control device in accordance with an embodiment of the present invention, the controller may be configured to form an electric field in the internal space through the electrode unit by generating the electric signal, in the first section in which the external module and the internal module move by the first control signal.

Further, in the effective gravity and electric field control device in accordance with an embodiment of the present invention, the controller may be configured to form an electric field in the internal space through the electrode unit by generating the electric signal, in the second section in which the external module and the internal module move by the second control signal.

Further, in the effective gravity and electric field control device in accordance with an embodiment of the present invention, the internal module may include a gravity measuring unit configured to measure an effective gravity applied to the internal module.

Further, in the effective gravity and electric field control device in accordance with an embodiment of the present invention, the controller may be configured to generate an electric signal to be applied to the internal module based on the effective gravity of the internal module measured by the gravity measuring unit.

In addition, in the effective gravity and electric field control device, the controller may be configured to perform control to form an electric field and current distribution from the electrode unit to the internal space when the internal module is in a weighted gravity state based on the signal measured from the gravity measuring unit.

In addition, in the effective gravity and electric field control device, the controller may be configured to perform control to form an electric field and current distribution from the electrode unit to the internal space when the internal module is in a microgravity or zero gravity state based on the signal measured from the gravity measuring unit.

In an effective gravity and electric field control device of the present invention, as a method for increasing cell membrane permeability, concepts of gravitoporation and electro-gravitoporation, which are totally new methods, are presented.

In addition, according to the effective gravity and electric field control device of the present invention, by inducing a zero gravity, microgravity, low gravity, or weighted gravity state in a module in which a target cell is accommodated, it is possible to cause cell shape deformation and cell membrane perforations while minimizing unintended cell destruction.

In addition, according to the effective gravity and electric field control device of the present invention, by inducing a change in gravity in the target cell or combining a method for applying an electric field, it is possible to effectively increase cell membrane permeability and it is possible to insert specific substances into a large number of target cells in a short period of time.

In addition, according to the effective gravity and electric field control device of the present invention, by designing a dual module structure in which an external module protects an internal module in which the target cell, which is the experimental body, is accommodated, there is an advantage in that more stable experiments can be performed.

In addition, according to the effective gravity and electric field control device of the present invention, by controlling a relative position of the internal module by controlling the movement of the external module even in an environment where the external module slows down due to air resistance, there is an advantage in that the zero gravity or low gravity state of the internal module accommodated within the external module can be stably maintained for as long as possible.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1 illustrates an overall configuration of an effective gravity and electric field control device of the present invention;

FIG. 2 illustrates a schematic block diagram of the effective gravity and electric field control device of the present invention;

FIG. 3 illustrates a structure of a dual module included in the effective gravity and electric field control device of the present invention;

FIG. 4 illustrates a structure of an internal module included in the dual module;

FIG. 5 illustrates a cross-section of the dual module included in the effective gravity and electric field control device of the present invention;

FIG. 6 shows a graph of a relative speed of the internal module and an external module over time when the effective gravity and electric field control device of the present invention implements zero gravity or microgravity;

FIG. 7 shows a graph of the relative speed of the internal module and the external module over time when the effective gravity and electric field control device of the present invention implements zero gravity or microgravity;

FIG. 8 schematically illustrates movement states of the internal module and the external module observed from the outside when the effective gravity and electric field control device of the present invention implements zero gravity or microgravity;

FIG. 9 is a diagram illustrating changes in cell membranes of target cells loaded in the internal module when zero gravity or microgravity is implemented using the effective gravity and electric field control device of the present invention;

FIG. 10 is a diagram illustrating increase in cell membrane permeability of target cells loaded in the internal module when zero gravity or microgravity is implemented using the effective gravity and electric field control device of the present invention;

FIG. 11 schematically illustrates another embodiment of an internal module included in the effective gravity and electric field control device of the present invention;

FIG. 12 schematically illustrates a configuration of an electric field control device connected to the internal module of FIG. 11;

FIG. 13 is a diagram for describing a electric field formed in the internal module of FIG. 11 and a resulting change in cell membrane permeability;

FIG. 14 is a diagram for describing one embodiment of increasing cell membrane permeability of target cells by combining a change in gravity and an electric field application method through the effective gravity and electric field control device of the present invention; and

FIG. 15 is a diagram for describing another embodiment of increasing cell membrane permeability of target cells by combining a change in gravity and an electric field application method through the effective gravity and electric field control device of the present invention.

### DETAILED DESCRIPTION OF THE PREFERRED EMBODIMENTS

Advantages and features of the present invention, and methods for achieving the advantages and features will be clarified with reference to embodiments described below in detail together with the accompanying drawings. However, the present invention is not limited to the embodiments disclosed below, but will be implemented in a variety of different forms. The present embodiments are only provided to allow the present invention to be complete, and to completely inform those skilled in the art of the scope of the invention, and the present invention is merely defined by scope of the claims.

Although first, second, etc. are used to describe various components, these components are of course not limited by these terms. These terms are merely used to distinguish one component from another. Therefore, it goes without saying that a first component mentioned below may also be a second component within the technical spirit of the present invention.

In the following embodiments, terms such as include or have mean the presence of features or components described in the specification, and are not intended to exclude in advance the possibility of adding one or more other features or components.

In drawings, for convenience of description, the sizes of components may be exaggerated or reduced. For example, the size and shape of each component in the drawings are arbitrarily illustrated for convenience of description, the present invention is not limited thereto.

Like reference numerals refer to like elements throughout the specification.

Features of various embodiments of the present invention may be partially or overall coupled to or combined with each other, and may be variously inter-operated with each other and driven technically as those skilled in the art can sufficiently understand. The embodiments of the present invention may be carried out independently from each other, or may be carried out together in co-dependent relationship.

In an effective gravity and electric field control device 1000 of the present invention, as a method for increasing cell membrane permeability, concepts of gravitoporation and electro-gravitoporation, which are significantly improved methods compared to methods in the related art, are to be presented. Gravitoporation is a method that uses a change in gravity as a principle of modifying cell morphology, temporarily increasing permeability of a cell membrane, and injecting genes through this increasing. Electro-gravitoporation is a method obtained by combining the gravitoporation with a method for applying an electric field, and is a new method that improves limitations of the existing electroporation.

Hereinafter, with reference to the attached drawings, the effective gravity and electric field control device 1000 and a control method on gravitoporation and electro-gravitoporation according to the present invention will be described in detail.

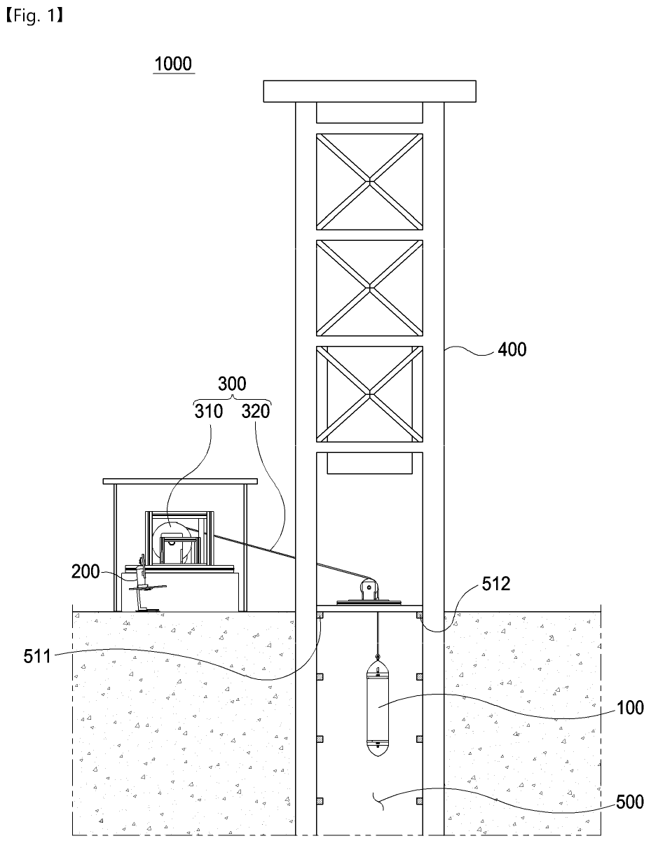

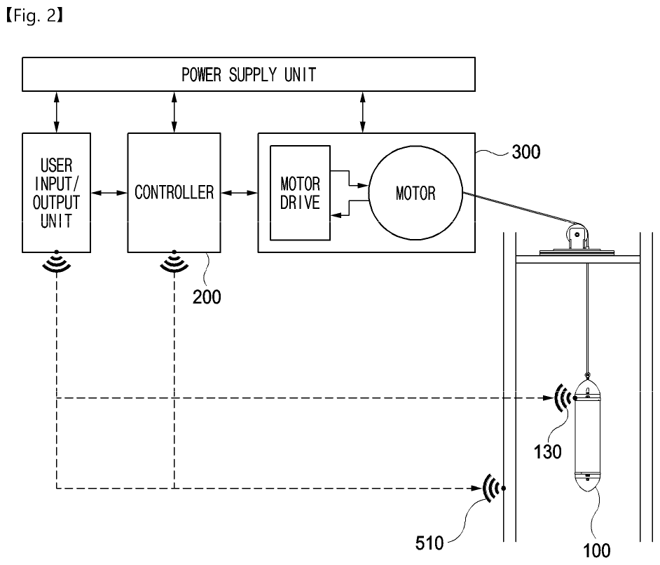

FIG. 1 illustrates an overall configuration of the effective gravity and electric field control device 1000 of the present invention, and FIG. 2 illustrates a schematic block diagram of the effective gravity and electric field control device 1000 of the present invention.

Referring to FIG. 1, the effective gravity and electric field control device 1000 of the present invention is a device that implements a change in gravity using a dual module 100. Specifically, the effective gravity and electric field control device 1000 of the present invention is a device that implements a zero gravity, microgravity, low gravity, or weighted gravity state of an internal module 120 mounted in a moving external module 110. Hereinafter, “change in gravity” are meant to encompass all states of zero gravity, microgravity, low gravity, or weighted gravity.

The effective gravity and electric field control device 1000 of the present invention includes the dual module 100, a controller 200, an actuator 300, and a support body 400. In addition, the effective gravity and electric field control device 1000 of the present invention may additionally include a user input/output unit and a power supply unit as illustrated in FIG. 2.

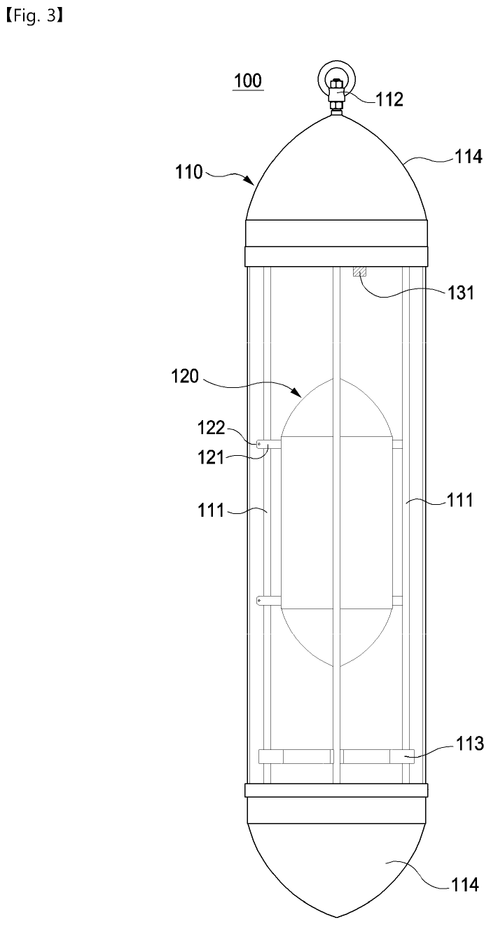

The dual module 100 is a module that implements the change in gravity. A structure of the dual module 100 will be described in detail with reference to FIGS. 3 to 5 below.

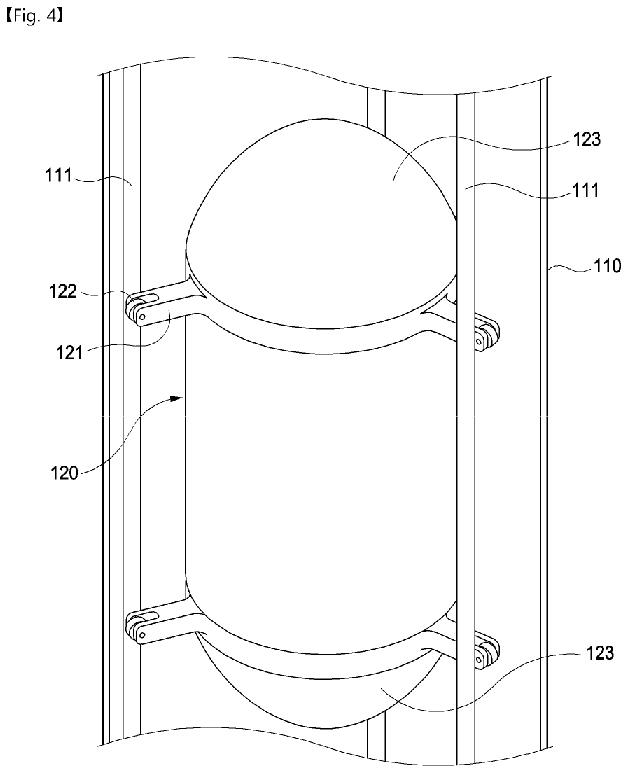

FIG. 3 illustrates a structure of the dual module 100 included in the effective gravity and electric field control device 1000 of the present invention, FIG. 4 illustrates a structure of the internal module 120 included in the dual module 100, and FIG. 5 illustrates a cross-section of the dual module 100 included in the effective gravity and electric field control device 1000 of the present invention.

Referring to FIG. 3, the dual module 100 includes the external module 110 and the internal module 120 mounted inside the external module 110.

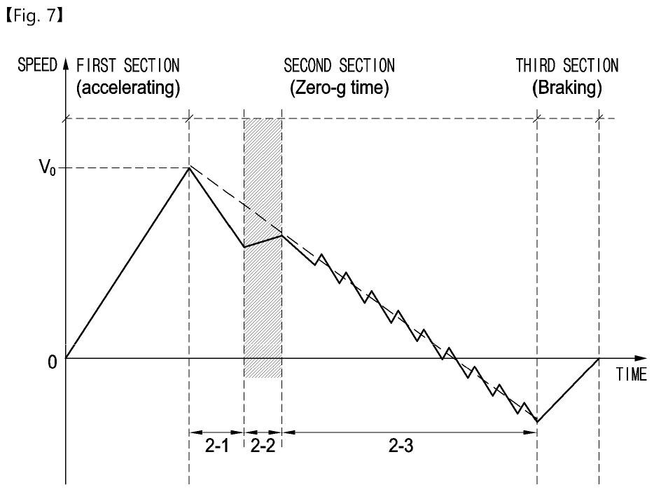

The external module 110 is provided with a movement space the internal module 120 therein. The external module 110 is a module of which its movement is controlled by the actuator 300. The external module 110 controls at least one physical quantity related to the movement by the actuator 300. Here, the physical quantity related to the movement of the external module 110 may be a position or moving speed of the external module 110. A specific control method in which the external module 110 is controlled by the actuator 300 will be described below with reference to FIGS. 7 to 10.

The effective gravity and electric field control device 1000 of the present invention may include, as the actuator 300, a motor 310 and a string 320 that are connected to the external module 110 to move the external module 110. Hereinafter, the actuator 300 will be described based on the motor 310 and the string 320.

The external module 110 includes a support member 111.

The support member 111 defines a movement space, which is a movement space of the internal module 120, inside the external module 110 and supports a shape of the external module 110.

The support member 111 is a bar-shaped member disposed along a longitudinal direction of the external module 110. The support member 111 is a bar-shaped member that connects upper and lower parts of the external module 110.

Here, the external module 110 may be formed so that a load of the lower part of the external module 110 is greater than a load of the upper part of the external module 110. When the external module 110 falls in a fall space 500, inappropriate movements such as the upper and lower parts of the external module 110 being reversed or rotated may occur due to air resistance applied to the external module 110. In order to prevent this problem, it is preferable that the load of the lower part of the external module 110 is greater than the load of the upper part of the external module 110, and it is preferable that the support member 111 is connected to the upper part thereof while bearings the load of the lower part of the external module 110.

The external module 110 includes a connection member 112 to which the string 320 is connected at least on one side.

The external module 110 receives an external force from the actuator 300 through the connection member 112. The connection member 112 may be installed in a central portion of an upper end of the external module 110. The connection member 112 may be formed in a ring shape so that the string 320 is able to be connected thereto.

The connection member 112 may include a bearing therein. The connection member 112 may include a bearing member that functions to prevent rotation or twist of the string 320 from being transmitted to the external module 110 or to prevent rotation of the external module 110 from being transmitted to the string 320. In other words, the connection member 112 may include a member such as a bearing so that an inappropriate movement of the string 320 or the external module 110 does not affect each other.

The external module 110 may include a shock absorbing member 113.

The shock absorbing member 113 is provided at at least one end inside the external module 110 and absorbs shock resulting from the movement of the internal module 120. Specifically, the shock absorbing member 113 is a component for reducing impact of the internal module 120 on the external module 110 by when the internal module 120 moves up or down within a movement space of the external module 110.

The shock absorbing member 113 may be used to connect a plurality of support members 111 and may have a ring shape. As an example, as an inner cap member 123, which will be described below, touches the ring-shaped shock absorbing member 113, the impact of the internal module 120 on the external module 110 may be reduced. The shock absorbing member 113 or the inner cap member 123 is preferably made of an elastic material.

Meanwhile, in FIG. 3, the shock absorbing member 113 is illustrated as being installed only on the lower side of the external module 110, but the shock absorbing member 113 is not limited thereto and may, of course, be installed on the upper side of the external module 110.

The external module 110 may include an outer cap member 114.

The outer cap member 114 is provided at at least one end outside the external module 110 and reduces air resistance due to the movement of the external module 110. Specifically, the outer cap member 114 is formed at both ends of the external module 110 and is a component for minimizing air resistance acting on the external module 110 when the external module 110 moves up or down. The above-described connection member 112 may be formed at an end of the outer cap member 114.

Meanwhile, when the external module 110 falls in the fall space 500, in order to prevent inappropriate movements from occurring, such as the upper and lower parts of the external module 110 being reversed or rotated, an object that applies a load may be accommodated in the outer cap member 114 provided at a lower end of the external module 110. For example, the object that applies the load may be a structure such as a battery mounted on the dual module 100.

The external module 110 may include a position detection sensor 131 for detecting a position of the internal module 120.

The position detection sensor 131 is provided at at least one inner end of the external module 110 and is a means for detecting the position of the internal module 120 within the movement space. Here, the position detection sensor 131 may be a LiDAR sensor for measuring position coordinates of a reflector, that is, the internal module 120, by shooting a laser pulse and measuring the time it takes for the laser pulse to be reflected and returned. In FIG. 3, the position detection sensor 131 is illustrated as being installed on the upper side of the external module 110, but the position detection sensor 131 is not limited thereto and may, of course, be installed on the lower side of the external module 110.

The dual module 100 includes the internal module 120 mounted inside the external module 110.

The internal module 120 is a module that moves independently from the external module 110 within the movement space of the external module 110 by the external force transmitted through the external module 110.

The internal module 120 is provided with an internal space for transporting objects. Here, the object may be an object that performs an experiment through the change in gravity, and the object may include various types. For example, the internal module 120 may accommodate anything from cells-level objects to objects corresponding to animals or humans.

The internal module 120 includes a plurality of protruding members 121 extending toward an inner wall of the external module 110.

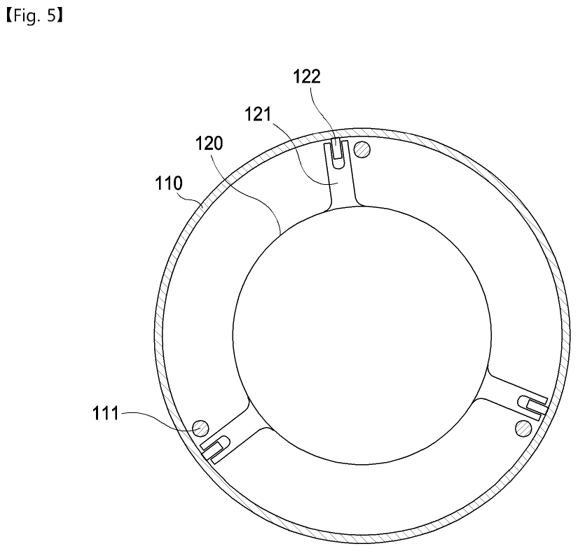

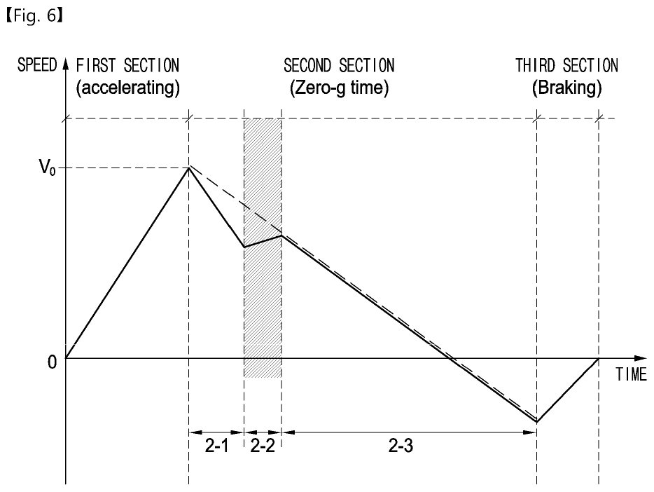

As illustrated in FIG. 6, the protruding members 121, together with the support member 111 of the external module 110, may restrict the internal module 120 from rotating beyond a threshold value within the movement space of the external module 110. Even when the internal module 120 rotates within the movement space, the protruding members 121 may prevent the internal module 120 from going beyond the position where the support member 111 is installed, and thereby the internal module 120 may be restricted from rotating beyond a predetermined angle.

The internal module 120 includes rollers 122 provided at ends of the respective protruding members 121.

The roller 122 is a component for reducing friction caused by contact between the external module 110 and the internal module 120. The roller 122 is disposed to extend from the internal module 120 and contact the inner wall of the external module 110. When the internal module 120 touches the external module 110 during movement, the roller 122 may minimize the action of unnecessary external force such as friction force acting on the internal module 120. It is desirable that the internal module 120 moves smoothly along the inner wall of the external module 110.

In describing the present invention, the roller 122 is exemplified, but of course, various means other than the roller 122 may be applied as long as the means is a component for reducing friction between the internal module 120 and the external module 110. For example, the roller 122 may be a roller bearing member that performs one-dimensional linear motion as illustrated in FIGS. 4 and 5, or may be a ball bearing member that performs multi-angular rotational motion.

The internal module 120 may include a gravity measuring unit 125 for measuring the effective gravity state of the internal module. The gravity measuring unit 125 is a sensor for measuring the effective gravity applied to the internal module 120 and may include an acceleration sensor and a gyro sensor.

Meanwhile, the support body 400 is a device for providing a fall space 500 where the dual module 100 moves. The support body 400 may be installed on the ground or a building to form the fall space 500 in which the dual module 100 moves. As illustrated in FIG. 1, the support body 400 may be a facility extending upward and downward from a reference surface. FIG. 1 illustrates an example where the support body 400 extends underground based on the ground to form a vertical space in which the dual module 100 moves and extends above the ground to provide an installation space for the effective gravity and electric field control device 1000.

Various sensor units 510 for monitoring the movement of the dual module 100 may be disposed in the fall space 500.

The sensor units 510 include a position detection sensor 511 for detecting the movement or position of the dual module 100, especially the external module 110. In addition, the sensor units 510 may include a camera 512 for acquiring an image to monitor the movement or position of the external module 110. The sensor units 510 installed in the fall space 500 may include an infrared sensor and a temperature sensor as well as various sensors such as the position detection sensor 131 and the camera 512.

Hereinafter, with reference to FIGS. 6 to 8, a control method for implementing the change in gravity by the effective gravity and electric field control device 1000 of the present invention will be described in detail. Hereinafter, description will be made with reference to FIGS. 1 to 5 described above together.

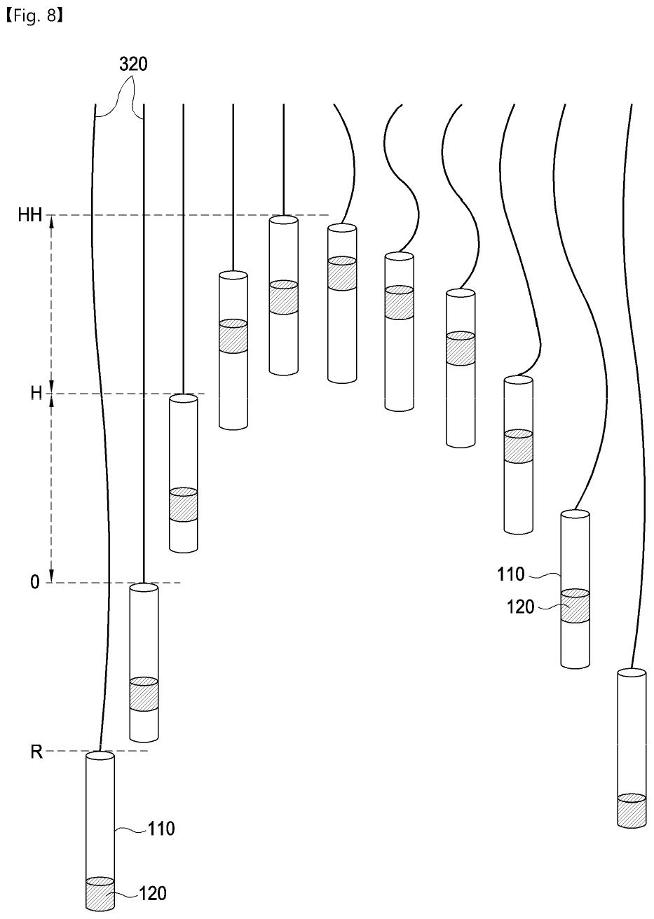

FIGS. 6 and 7 show graphs of a relative speed of the external module 110 and the internal module 120 over time when the effective gravity and electric field control device 1000 of the present invention implements zero gravity or microgravity, and FIG. 8 schematically illustrates movement states of the external module 110 and the internal module 120 observed from the outside when the effective gravity and electric field control device 1000 of the present invention implements zero gravity or microgravity.

The controller 200 generates a control signal of the actuator 300 for controlling the movement of the external module 110. The controller 200 generates a control signal for controlling at least one physical quantity related to the movement of the external module 110.

The controller 200 may receive data about the external module 110 and the internal module 120 from the above-described sensor units 130 and 510 and generate a control signal for controlling the movement of the external module 110 based on the received data. The controller 200 may receive data about the positions of the external module 110 and the internal module 120 from the position detection sensors 131 and 511 and the camera 512 and generate a control signal for controlling the movement of the external module 110 based on the received position data for the external module 110 and the internal module 120.

Meanwhile, in addition to the position detection sensor 131, the controller 200 may receive data about the state of the dual module 100 through the sensor unit 130 mounted on the dual module 100 and the sensor unit 510 installed in the fall space 500. For example, when the controller 200 determines that an event such as device overheating, abnormal movement detection, shock occurrence detection, or the like, has occurred through the sensor units 130 and 510, the controller 200 may generate a control signal such as emergency braking or the like.

The actuator 300 moves the external module 110 so that the external module 110 has at least one physical quantity according to the control signal received from the controller 200. The actuator 300 may include the motor 310, a motor drive for controlling the motor, and the string 320 connected to the motor 310.

Meanwhile, in describing the effective gravity and electric field control device 1000 of the present invention, the actuator 300 is described with the motor 310 and the string 320, but the scope of the present invention is not limited thereto, and as the actuator 300, of course, a pneumatic actuator, a hydraulic actuator, a rail structure, a magnetic levitation method, or the like, may be applied.

The physical quantity related to the movement of the external module 110 may be the speed of the external module 110, the position of the external module 110, the relative position of the external module 110 and the internal module 120, or the relative speed of the external module 110 and the internal module 120. FIGS. 6 and 7 illustrate the movement state of the dual module 100 of the present invention from the perspective of controlling the relative speed of the external module 110 and the internal module 120, and FIG. 8 illustrates the movement state of the dual module 100 of the present invention from the perspective of controlling the relative distance between the external module 110 and the internal module 120.

In the present invention, as illustrated in FIG. 8, the controller 200 generates a control signal for controlling the gravity of the dual module 100 rising from a bottom of the fall space 500 formed by the support body 400, starting from the bottom. The controller 200 applies tension to the string 320 connected to the upper side of the external module 110 so that the external module 110 and the internal module 120 rise vertically upward from the bottom, which is a starting point.

Referring to FIGS. 6 and 7, the controller 200 may generate control signals for accelerating the dual module 100 (corresponding to a first section), inducing the change in gravity (corresponding to a second section), and braking (corresponding to a third section). Specifically, the controller 200 generates a first control signal in the first section for accelerating the dual module 100. The controller 200 generates a second control signal in the second section in which a change in gravity, for example, zero gravity (zero-g) or microgravity, acts on the internal module 120 of the dual module 100. In addition, the controller 200 generates a third control signal in the third section in which the dual module 100 is braking.

Hereinafter, a method for controlling the movement of the external module 110 and the internal module 120 in the first section will be described.

The controller 200 generates the first control signal for controlling the actuator 300 so that an external force is applied to the internal module 120 through the external module 110 in the first section.

The first control signal is a control signal for pulling the string 320 connected to the external module 110 with the force or torque of the motor. In the first section, which is an acceleration section, by this first control signal, the external module 110 and the internal module 120 move up from the bottom as one unit.

As illustrated in FIGS. 6 and 7, in the first section, the speed of the external module 110 (shown by a solid line) and the speed of the internal module 120 (shown by a dotted line) may have the same speed and accelerate. In the first section, the external module 110 and the internal module 120 move up to a preset speed Vo by the actuator 300. In the first section, the relative speed of the internal module 120 and the external module 110 may be less than a first threshold speed, where the first threshold speed may substantially correspond to zero. In other words, the relative speed of the internal module 120 and the external module 110 in the first section may be zero.

As illustrated in FIG. 8, in the first section, the external module 110 and the internal module 120 move up from the bottom (not illustrated) to a point “R”. At this time, the internal module 120 is positioned in a seated state on the lower side of the external module 110, and the internal module 120 moves up together with the external module 110 by the external force applied through the external module 110. In the first section, the relative distance of the internal module 120 and the external module 110 may be less than a first threshold distance, where the first threshold distance may substantially correspond to zero. In other words, the relative distance of the internal module 120 and the external module 110 in the first section may be zero.

Hereinafter, a method for controlling the movement of the external module 110 and the internal module 120 in the second section will be described.

First, the second control signal in the second section is a combination of various control signals for inducing a change in effective gravity in the internal module 120 mounted within the external module 110.

The second section, which is an effective gravity control section, may include a 2-1 section in which the internal module 120 is separated from the external module 110, a 2-2 section in which the speed or position of the external module 110 is corrected based on the position data about the internal module 120, or a 2-3 section in which the external module 110 follows the speed or position of the internal module 120.

First, a method for controlling the movement of the external module 110 and the internal module 120 in the 2-1 section will be described.

The controller 200 generates a 2-1 control signal for separating the internal module 120 from the external module 110. The controller 200 generates a signal for causing the internal module 120 to initiate a movement independently from the external module 110 in the 2-1 section, which is the moment when the first section enters the second section.

The 2-1 control signal is a motor control signal for separating the internal module 120 from the external module 110. The 2-1 control signal may be a signal for releasing the external force applied to the internal module 120 through the external module 110 at the moment when the dual module 100 reaches a preset speed or at the moment when the dual module 100 reaches a preset position. In other words, the 2-1 control signal may be a signal for temporarily releasing the force or torque of the motor 310 applied to the string 320 connected to the external module 110 at the moment when the dual module 100 reaches the preset speed or at the moment when the dual module 100 reaches the preset position.

As illustrated in FIGS. 6 and 7, in the 2-1 section, the speed of the external module 110 (shown by a solid line) and the speed of the internal module 120 (shown by a dotted line) gradually become different. Due to external forces such as air resistance applied to the external module 110, the speed of the external module 110 decelerates faster than the speed of the internal module 120. The difference between the speed of the external module 110 and the speed of the internal module 120 gradually increases. The movement in the 2-1 section is maintained until the relative speed of the internal module 120 and the external module 110 reaches a preset second threshold speed. The movement in the 2-1 section is maintained until the speed difference between the internal module 120 and the external module 110 reaches the preset second threshold speed. Here, the second threshold speed may correspond to a difference between the dotted line and the solid line at a boundary between the 2-1 section and the 2-2 section.

The point where the 2-1 section is entered is shown as the point “R” in FIG. 8. In this case, the internal module 120, which is seated on the lower side of the external module 110, moves up independently from the external module 110. Upon entering the second section after the first section, separation of the internal module 120 from the external module 110 begins.

In the 2-1 section, the internal module 120 moves vertically upward within the external module 110 due to inertia according to the external force transmitted from the external module 110. The 2-1 section corresponds to a section in which the external module 110 rises from the point “R” to the point “0” in FIG. 8. In the 2-1 section, the positions of the external module 110 and the internal module 120 gradually become different. Due to external forces such as air resistance applied to the external module 110, the rising speed of the external module 110 decelerates faster than the rising speed of the internal module 120. The difference between the position of the external module 110 and the position of the internal module 120 gradually increases. The movement in the 2-1 section is maintained until the relative distance between the internal module 120 and the external module 110 reaches a preset second threshold distance. Here, the second threshold distance may be a distance from the bottom of the external module 110 to the bottom of the internal module 120, or a distance from the top of the external module 110 to the top of the internal module 120.

In the 2-1 section, the external module 110 performs a decelerated movement relative to the ground. At this time, a force applied to the external module 110 may be the sum of gravity and air resistance. The internal module 120 may perform an ideal parabolic movement relative to the ground. The ideal parabolic movement may be a parabolic movement in a vacuum or a movement close to the parabolic movement in vacuum. In this case, it is preferable that the force applied to the internal module 120 is gravity. A slope of a speed graph (shown by a dotted line) of the internal module 120 preferably has a slope value corresponding to the acceleration of gravity. However, a case where there is a slight air resistance applied to the internal module 120 or the like is not excluded from the scope of the present invention.

Next, a method for controlling the movement of the external module 110 and the internal module 120 in the 2-2 section will be described.

The controller 200 generates a 2-2 control signal for correcting the speed or position of the external module 110. The controller 200 generates a signal for correcting the position of the external module 110 based on the position data about the internal module 120 in the 2-2 section after the 2-1 section.

The 2-2 control signal is a motor control signal for causing the external module 110 to keep up with the movement of the internal module 120. The 2-2 control signal may be a signal for greatly applying tension of the string 320 to the external module 110. In other words, the 2-2 control signal may be a signal for temporarily applying a large force or torque of the motor 310 to the string 320 connected to the external module 110 at the moment when the speed difference between the internal module 120 and the external module 110 reaches the preset second threshold speed or at the moment when the position difference between the internal module 120 and the external module 110 reaches the preset second threshold distance.

As illustrated in FIGS. 6 and 7, in the 2-2 section, the difference between the speed of the external module 110 (shown by a solid line) and the speed of the internal module 120 (shown by a dotted line) gradually decreases. In order to compensate for an external force such as air resistance applied to the external module 110 and the like, when tension is momentarily applied to the external module 110, the speed of the external module 110 temporarily accelerates. At this time, the difference between the speed of the external module 110 and the speed of the internal module 120 gradually decreases. Until the relative speed of the internal module 120 and the external module 110 reaches 0 (zero) or until the relative speed of the internal module 120 and the external module 110 reaches a preset third threshold speed, the movement in the 2-2 section is maintained. Here, the third threshold speed may correspond to a difference between the dotted line and the solid line at a boundary between the 2-2 section and the 2-3 section.

In the 2-2 section, the external module 110 moves up more than the internal module 120 moves up. In other words, in the 2-2 section, the external module 110 moves to catch up with the internal module 120. The 2-2 section corresponds to a section in which the external module 110 rises from the point “0” to a point “H” in FIG. 8. The 2-2 section is a section in which the tension of the string 320 applied to the external module 110 is maintained. In the 2-2 section, the difference between the position of the external module 110 and the position of the internal module 120 may gradually decrease. By the tension overcoming the air resistance applied to the external module 110, the rising speed of the external module 110 accelerates faster than the rising speed of the internal module 120. The movement in the 2-2 section is maintained until the relative distance between the internal module 120 and the external module 110 reaches a preset third threshold position. Here, the third threshold position may be the distance from the bottom of the external module 110 to the bottom of the internal module 120, or the distance from the top of the external module 110 to the top of the internal module 120.

Next, a method for controlling the movement of the external module 110 and the internal module 120 in the 2-3 section will be described.

The controller 200 generates a 2-3 control signal for causing the external module 110 to follow the speed or position of the internal module 120. The controller 200 generates a feedback control signal for causing the external module 110 to follow the position of the internal module 120 based on the position data about the internal module 120 in the 2-3 section after the 2-2 section.

The 2-3 control signal is a motor control signal for causing the external module 110 to follow the movement of the internal module 120. The 2-3 control signal may be a signal for feedback-controlling the tension of the string 320 applied to the external module 110 based on the position or speed of the internal module 120. In other words, the 2-3 control signal may be a feedback control signal for causing the external module 110 to follow the position of the internal module 120 at the moment when the speed difference between the internal module 120 and the external module 110 reaches the preset third threshold speed or at the moment when the position difference between the internal module 120 and the external module 110 reaches the preset third threshold position.

As illustrated in FIGS. 6 and 7, in the 2-3 section, the speed of the external module 110 (shown by a solid line) and the speed of the internal module 120 (shown by a dotted line) may be substantially the same. When tension is applied to the external module 110 to compensate for the external force such as air resistance, the speed of the external module 110 may have a speed similar to a state of the internal module 120 maintaining an ideal parabolic movement state.

Here, the reference to the speed of the external module 110 (shown by the solid line) and the speed of the internal module 120 (shown by the dotted line) being substantially the same may refer to a state in which the relative speed of the internal module 120 and the external module 110 is substantially 0 (zero) as illustrated in FIG. 6. In contrast, the reference to the speed of the external module 110 (shown by the solid line) and the speed of the internal module 120 (shown by the dotted line) being substantially the same may refer to a state in which, even when there is a slight difference in the relative speed of the internal module 120 and the external module 110, the speed of the external module 110 is continuously adjusted to the speed of the internal module 120 as illustrated in FIG. 7. In other words, the reference to the speed of the external module 110 (shown by the solid line) and the speed of the internal module 120 (shown by a dotted line) being substantially the same includes the state in the 2-3 section illustrated in FIG. 6 and the state in the 2-3 section illustrated in FIG. 7.

In the 2-3 section, movement is maintained until the internal module 120 and the external module 110 are substantially brought into touch. The movement in the 2-3 section may be maintained until the inner cap member 123 of the internal module 120 approaches the shock absorbing member 113 of the external module 110 by a preset distance.

The 2-3 section corresponds to a section in which the external module 110 rises from the point “H” to a point “HH” and then falls in FIG. 8. The 2-3 section is a section in which the tension of the string 320 applied to the external module 110 is maintained for a certain period and then released. It is preferable that the difference between the position of the external module 110 and the position of the internal module 120 in the 2-3 section is maintained within a preset distance range.

The movement of the external module 110 following the internal module 120 in the 2-3 section corresponds to a movement that is at least partially based on an ideal parabolic movement with respect to the ground. This movement is an ideal parabolic movement in which the internal module 120 falls due to gravity, while the movement of the external module 110 is a movement implemented by feedback control by the controller 200 and the actuator 300. Here, the ideal parabolic movement may be a parabolic movement in a vacuum or a movement close to the parabolic movement in vacuum.

Therefore, through the control method by the controller 200 in the second section, an advantage of increasing a flight time (that is, net fall time) of the internal module is gained. Through the control method by the controller 200 in the second section, even in environments where the external module 110 slows down due to air resistance, by controlling the relative positions of the internal module 120 and the external module 110, the ideal parabolic movement state of the internal module 120 accommodated within the external module 110 may be stably maintained for as long as possible.

Hereinafter, a method for controlling the movement of the external module 110 and the internal module 120 in the third section will be described.

The controller 200 generates a third control signal for braking the external module 110 and the internal module 120 in the third section. The third control signal is a control signal for pulling the string 320 connected to the external module 110 with the force or torque of the motor. By this third control signal, the external module 110 and the internal module 120, which are accelerating, collectively perform a movement to reduce their speed.

As illustrated in FIGS. 6 and 7, in the third section, the speed of the external module 110 (shown by a solid line) and the speed of the internal module 120 (shown by a dotted line) may have the same speed and decelerate. In the third section, the external module 110 and the internal module 120 are brought to a stop by the actuator 300. At this time, since the external module 110 and the internal module 120 are substantially in contact, the relative speed of the internal module 120 and the external module 110 in the third section may be zero.

Referring to FIG. 8, the third section may represent a movement state (not illustrated) after a point where the external module 110 and the internal module 120 meet again (a final state illustrated in FIG. 9).

Hereinafter, with reference to FIGS. 9 and 10, the gravitoporation performed by the effective gravity and electric field control device 1000 of the present invention will be described in detail.

FIGS. 9 and 10 are diagrams illustrating changes in cell membranes and increase in cell membrane permeability of target cells when zero gravity or microgravity is implemented using the effective gravity and electric field control device 1000 of the present invention. Specifically, FIG. 9 is a diagram illustrating changes in the cell membrane of target cells loaded in the internal module, and FIG. 10 is a diagram illustrating increase in cell membrane permeability of target cells loaded in the internal module. Description will be made with reference to FIGS. 1 to 8 described above together.

As described above, the internal module 120 is a module that moves independently from the external module 110 by an external force transmitted through the external module 110. The internal module 120 is provided with an internal space for transporting an object, and the object may be an object on which an experiment is performed through the change in gravity.

In describing the following embodiment, the internal module 120 may include target cells C as objects and specific substances D to be transfected into the target cells. The internal module 120 includes an internal space 124 in which the target cells C and the specific substances D to be transfected into the target cells are loaded in a suspension state.

FIG. 9 illustrates, stage by stage, an example of cell deformation when the change in gravity is induced by the effective gravity and electric field control device 1000 of the present invention in a state in which the target cells C are loaded. Stage (a) in FIG. 9 corresponds to a cell state in an initial stage, stage (b) in FIG. 9 corresponds to a cell state in the first section (accelerating) of FIGS. 6 and 7 described above, and stages (c) and (d) in FIG. 9 correspond to a cell state in the second section (zero-g time) of FIGS. 6 and 7 described above.

Stage (a), which may be viewed as the “initial stage”, shows a state in which cells in suspension are floating. When the cells have a circular shape shown, this is a state in which the cell shape is maintained without significantly deviating from an ideal state. Although the target cells C are shown as a circle, this is only an exemplary shape, and the cells may be understood as maintaining the cells' unique shape.

Referring to FIG. 8, stage (b) shows a change in cell state in the first section in which the external module 110 and the internal module 120 move up from the bottom to the point “R”. Here, the first section is an acceleration section in which the external module 110 and the internal module 120 accelerate. Here, the first section is a section in which the internal space 124 in which the target cells C are loaded accelerates.

When weighted gravity is applied to the target cells C accommodated in the internal space 124 by the effective gravity and electric field control device 1000 of the present invention, as illustrated in (b) of FIG. 9, the target cells C gradually become biased toward a lower part of the internal space 124. In this process, the shape of some target cells C may be transformed into an oval shape. In particular, when gravity greater than “g” is applied to the internal space 124, the target cells C may become increasingly deformed.

Then, referring to FIG. 8, stages (c) and (d) show the change in cell state in the second section in which the internal module 120 deviates from the lower side of the external module 110 and independently rises and falls after the point “R”. Here, the second section corresponds to a section in which the external module 110 rises from the point “R” to the point “HH” and then falls. The second section corresponds to a section in which the internal module 120 separates from the lower side of the external module 110 and independently rises and then falls again. In other words, the second section is a section in which the external module 110 and the internal module 120 are separated from each other and independently move and is a zero gravity or microgravity section in which zero gravity or microgravity acts on the internal module 120. Here, the second section is a section in which zero gravity or microgravity acts on the internal space 124 in which the target cells C are loaded.

When the internal space 124 is decelerated by the effective gravity and electric field control device 1000 of the present invention, as illustrated in (c) and (d) in FIG. 9, the target cells C gradually move to the upper part of the internal space 124 by inertia and become suspended. In this process, the deformed shape of the target cells C may be slightly recovered, and the cell membrane permeability may be increased in a process of this change in cell structure. When zero gravity or microgravity is applied to the target cells C accommodated in the internal space 124 by the effective gravity and electric field control device 1000 of the present invention, the cells may recover to the ideal cell shape such as the previous “initial stage”. In this process, the cell membrane permeability may be further increased, and this state of increased cell membrane permeability may be maintained for a certain period of time.

FIG. 10 illustrates, stage by stage, an example of increasing the cell membrane permeability when the change in gravity is induced by the effective gravity and electric field control device 1000 of the present invention in a state in which the target cells C and the specific substances D are loaded. That is, FIG. 10 is a diagram for describing a process in which the specific materials D, such as genetic material, are transfected into the target cells C.

As described in FIG. 9, stage (a) in FIG. 10 corresponds to a state in the initial stage, stage (b) in FIG. 10 corresponds to the stage in the first section in which the external module 110 and the internal module 120 move up from the bottom to the point “R”, and stages (c) and (d) in FIG. 10 correspond to the stage in the second section in which the internal module 120 separates from the lower side of the external module 110 and rises and falls independently.

Stage (a), which can be seen as the “initial stage”, and stage (b), in which the target cells C are deformed by acceleration, are a state in which the cell membrane permeability is low or a state in which there is no perforation in cell membranes, where the specific substances D having to be transfected into the target cells C exist outside the cell membranes.

Then, in a section where zero gravity or microgravity acts on the internal space 124, the shape of the deformed target cells C is recovered, and fine perforations are generated in the cell membranes and expand. In this case, the specific substances D existing outside the cell membranes of the target cells C penetrate and transfect into the cell membranes of the target cells C.

The effective gravity control method and the resulting method for increasing cell membrane permeability described in FIGS. 9 and 10 are referred to as “gravitoporation”, a new concept suggested by the present invention. According to this gravity perforation technology, in improving the cell membrane permeability, it is possible to effectively induce the change in cell shape while avoiding the method for directly applying energy to a cell membrane. Accordingly, according to the gravity perforation technology of the present invention, the specific substances D, such as genetic material, are inserted into a large number of target cells C in a short period of time while minimizing cell destruction. This has a great advantage in that it overcomes the limitations of existing electroporation and mechano-poration, which uses microchannels to inject genetic material into unit cells.

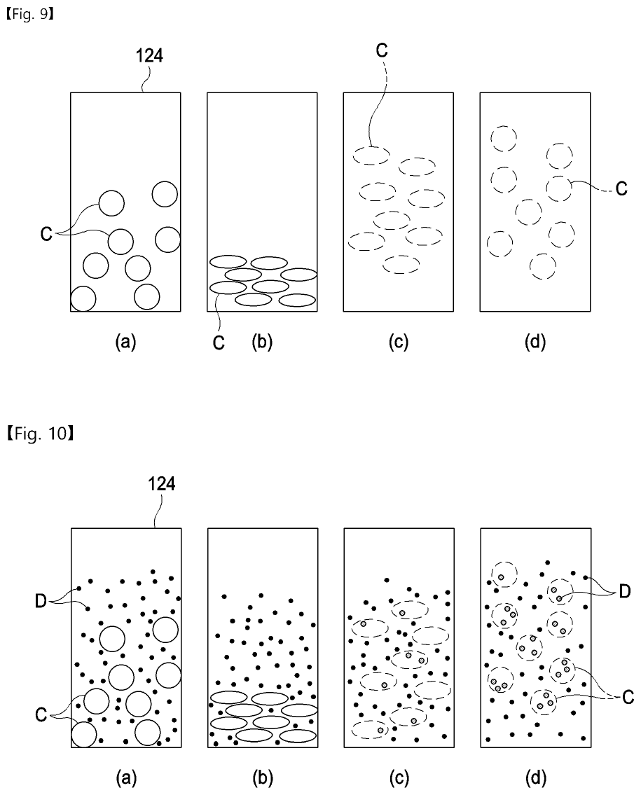

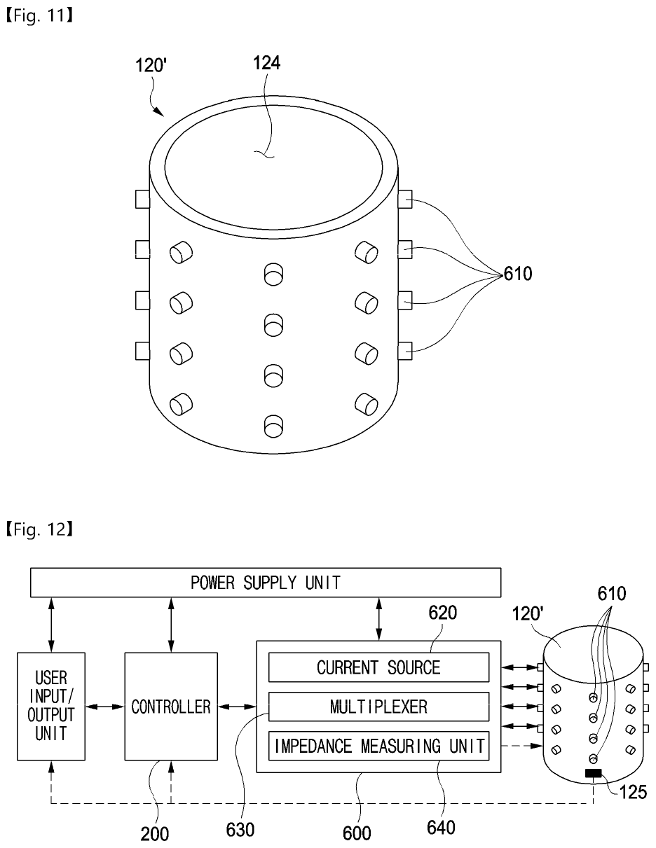

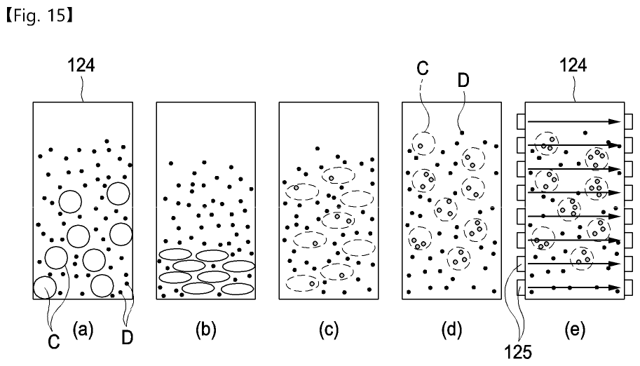

Hereinafter, with reference to FIGS. 11 and 15, the electro-gravitoporation performed by the effective gravity and electric field control device 1000 of the present invention will be described in detail.

The electroporation is a physical method capable of using electric pulses to create a temporary hole in a cell membrane and through the hole, transfecting a specific substance such as genetic material into a cell. Problems with the electroporation, such as unintended cell destruction in the process of applying electric pulses, or the like, are constantly being pointed out. However, when the electroporation is added to the gravitoporation using the effective gravity and electric field control device 1000 of the present invention, the cell damage problem may be solved and a large number of cells may be processed.

First, referring to FIGS. 11 to 13, a structure in which an electrode unit 600 is combined with an internal module 120′ and a method for forming an electric field accordingly will be described.

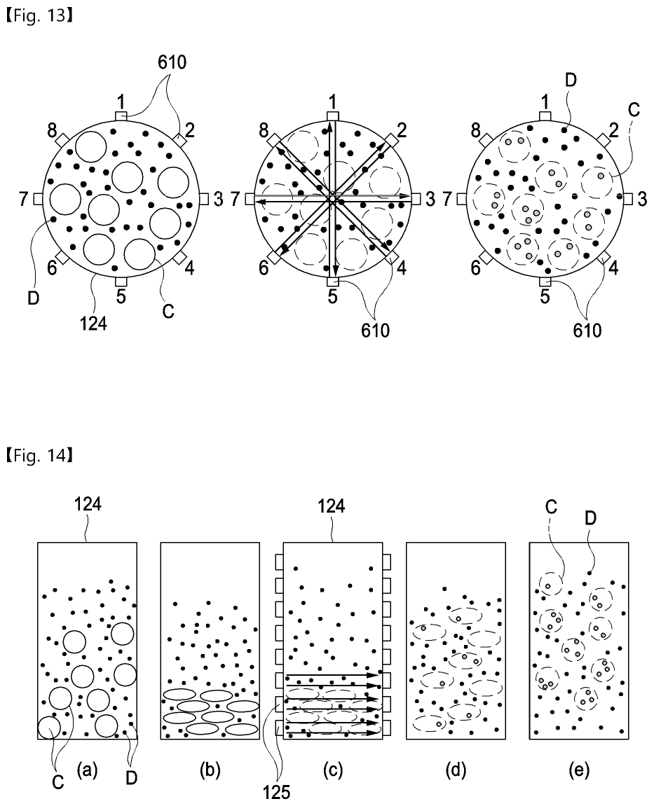

FIG. 11 schematically illustrates another embodiment of the internal module 120′ included in the effective gravity and electric field control device of the present invention, FIG. 12 schematically illustrates a configuration of an electric field control device connected to the internal module of FIG. 11, and FIG. 13 is a diagram for describing a electric field formed in an internal space 124 of the internal module 120′ of FIG. 11 and a resulting change in cell membrane permeability.

Referring to FIGS. 11 and 12, the effective gravity and electric field control device of the present invention includes the electrode unit 600 connected to the internal module 120′. The electrode unit 600 applies an electric field to the internal space 124 of the internal module 120′.

The electrode unit 600 includes a plurality of electrodes 610 installed along an outer wall of the internal module 120′. In addition, the electrode unit 600 may include a current source 620 and a multiplexer 630 connected to a plurality of electrodes 610.

The current source 620 supplies current to the electrodes, and the multiplexer 630 selectively distributes a signal applied from the current source 620 to a plurality of electrodes 610. The current source 620 and the multiplexer 630 may form current flow and material movement patterns according to the electric field applied to the internal space 124 where target cells C and specific substances D with electrical properties are accommodated through the plurality of electrodes 610 and a gradient of the electric field.

While an electric field application device in the related art mainly relies on only two parallel electrodes to form a uniform electric field distribution in a space where cells exist, the present invention may form a non-uniform electric field in the internal space 124 by including a plurality of electrodes 610, the current source 620, and the multiplexer 630. This non-uniform electric field may increase an effect of selectively delivering, into the target cells C, the specific substances D having electrical properties that move according to the changing gradient of the electric field.

Referring to FIG. 13 as one example of a method for forming an electric field, eight electrodes may be arranged at an angle of approximately 45 degrees to surround the internal space 124 where cells are accommodated. The electrodes illustrated in cross section in FIG. 13 may be in the form of an array as illustrated in FIG. 11, and the number and arrangement of electrodes illustrated should be understood as exemplary. The present invention is characterized by applying an electric field in various directions through a plurality of electrodes.

The plurality of electrodes may be paired with electrodes facing each other to form an electric field in the internal space 124. A method may be exemplified in which the electrodes are numbered 1 to 8 along an circumferential direction of the internal space 124, and + and − potentials are applied to electrodes in pair facing each other to form an electric field between facing electrodes. As illustrated in FIG. 13, a method may be exemplified in which electrode pairs (1, 5), (2, 6), (3, 7), and (4, 8) are sequentially activated and a rotating electric field is formed in the internal space 124. At this time, the above-described multiplexer 630 serves to simultaneously activate electrode pairs (1, 5), (2, 6), (3, 7), (4, 8), or a combination of several electrodes.

In this case, electrical stimulation or stress that causes perforations in various directions may be applied to the cell membranes of the target cells C existing in the internal space 124. According to the arrangement of the electrodes 610 of the present invention, it is possible to effectively improve the cell membrane permeability while using a lower intensity electric field compared to the existing electroporation method that forms a unidirectional electric field. Therefore, according to the arrangement of the electrodes 610 of the present invention, the present invention has the advantage of minimizing the destruction of normal cells in improving the cell membrane permeability.

This “rotation” type electric field application method may be simultaneously or sequentially performed through multi-layered electrodes of the module. For example, since a quadruple layer of electrodes is formed along the circumference of the internal module 120′ in FIG. 11, the quadruple layers of electrodes may be simultaneously synchronized to the operation of each of the numbered electrodes, or may operate individually. In addition, in addition to the rotation method illustrated in FIG. 13, a method may be exemplified in which + and − potentials are applied to adjacent electrode pairs so that adjacent electrods form an electric field along the circumference of the module. Although not illustrated, it is possible to exemplify a method in which electrode pairs (1, 2), (3, 4), (5, 6), and (7, 8) are sequentially activated to form an electric field along the outer wall of the module.

Besides, the method for activating a plurality of electrodes to form an electric field in the target cells C of the internal space 124 may be combined in various ways, and of course, electric fields of random directions and strengths may be formed in the cells inside the module by turning a plurality of electrodes on/off simultaneously or individually.

In addition, the electrode unit 600 may include an impedance measuring unit 640. By including the impedance measuring unit 640, the electrode unit 600 may measure the size and distribution of the electric field generated in the target cells C and the suspension, along with forming the electric field and applying the electric signal described above. The electrode unit 600 applies current to selected electrodes and measures the size and distribution of the electric field generated in the internal space 124 through the electrodes. By using this in-situ impedance measurement method, a delivery state of the specific substances D into the target cells C may be indirectly confirmed in real time, and through this, the advantage of being able to improve the delivery effect of the specific substances D may be gained.

Meanwhile, as described above, the internal module 120 may include a gravity measuring unit 125 for measuring the effective gravity state of the internal module. The controller 200 may control the operation of the electrode unit 600 based on the effective gravity of the internal module 120 measured by the gravity measuring unit 125. The controller 200 generates an electric signal through the electrode unit 600 based on the effective gravity of the internal module 120 measured by the gravity measuring unit 125.

The controller 200 generates an electric signal from the electrode unit 600 when the internal module 120 is in a weighted gravity state based on the signal measured from the gravity measuring unit 125. This is referred to as “preconditioning” and will be described in detail with reference to FIG. 14 below.

Further, the controller 200 generates an electric signal from the electrode unit 600 when the internal module 120 is in a microgravity or zero gravity state based on the signal measured from the gravity measuring unit 125. This is referred to as “enhanced transfection” and will be described in detail with reference to FIG. 15 below.

Various embodiments of electro-gravitoporation will be described with reference to FIGS. 14 and 15.

FIG. 14 is a diagram for describing an embodiment of “preconditioning” for increasing cell membrane permeability of target cells by combining a change in gravity and an electric field application method through the effective gravity and electric field control device of the present invention.

“Preconditioning” means a method for preconditioning target cells C by applying an electric field before causing perforations through a change in gravity in the target cells C or in an early stage when perforation is caused through the change in gravity. In this case, the controller 200 generates the electric signal and forms an electric field in the internal space 124, in the first section (acceleration section) in which the external module 110 and the internal module 120′ move by the first control signal.

Stages (a) and (b) in FIG. 14 correspond to stages (a) and (b) in FIG. 10. Stage (c) in FIG. 14, in which the electric field is applied, illustrates a situation in which the electric field is applied in stage (b), in which the target cells C are deformed by acceleration.

Stage (b) in FIG. 14 is a state in which perforations of the cell membranes is not formed solely by the change in gravity, but when the electric field is applied at this time, electrical stimulation or stress that causes perforations of the cell membranes may be applied. Then, when stages (d) and (e) in FIG. 14 corresponding to stages (c) and (d) in FIG. 10 are performed (that is, a microgravity stage is performed), the present invention has the advantage of significantly improving the cell membrane permeability compared to the pure gravitoporation without electroporation.

The electric field applied in stage (c) in FIG. 14 may be an electric field formed by a high frequency of 20 kHz or more, or an electric field generated by a low frequency of 1 kHz or less. When a high frequency is applied, the electric field may penetrate the cells and cause perforations in the cell membranes, and when a low frequency is applied, the electric field does not penetrate the cells, but while flowing along the cell membranes, may cause perforations in the cell membranes and increase the transfecting of genetic material into cells. Depending on a frequency range, there is a difference in whether the electric field may be applied to penetrate the cells or to flow along the epidermis, but the common characteristic is that they cause perforations of the cell membranes by the electric field before causing perforations of the cell membranes by microgravity.

According to the embodiment of “preconditioning”, the present invention has the advantage in that, by applying an electric field in the acceleration section of the internal space 124, a greater stress may be induced on the cell membrane of the target cells C, thereby promoting a cell deformation state, and then, when the deformed cell shape is recovered in the microgravity section, the cell membrane permeability may be significantly improved. In addition, since the change in gravity after applying the electric field causes perforations of the cell membranes, even when a low-intensity electric field is applied compared to the electric field applied in the existing electroporation, the degree of material penetration may be greatly improved. Therefore, it is possible to apply the electric field within a range of a critical value at which the target cells C are destroyed or below, and the present invention has the advantage of greatly increasing the cell membrane permeability while avoiding the problem of cell destruction that has been previously pointed out.

FIG. 15 is a diagram for describing an embodiment of “enhanced transfection” for increasing cell membrane permeability of target cells by combining a change in gravity and an electric field application method through the effective gravity and electric field control device of the present invention.

“Enhanced transfection” means a method for increasing the cell membrane permeability of target cells C and enhancing transfection of genetic material through the application of an electric field after cell membranes of the target cells C are weakened through a change in gravity or in a later stage when perforation is caused through the change in gravity. In this case, the controller 200 generates the electric signal and forms an electric field in the internal space 124, in the second section (zero gravity or microgravity section) in which the external module 110 and the internal module 120′ move by the second control signal.

Stages (a) to (d) in FIG. 15 correspond to stages (a) to (d) in FIG. 10. Stage (e) in FIG. 15, in which an electric field is applied, illustrates a situation in which the electric field is applied after the cell membranes are weakened or perforations are caused by the change in gravity.

Stage (e) in FIG. 15 serves to add electrical stimulation or stress that causes perforations in the cell membranes by applying the electric field after the cell membranes are perforated due to the change in gravity. In this way, the present invention has the advantage of significantly improving the cell membrane permeability compared to the pure gravitoporation without electroporation.

The electric field applied in stage (e) in FIG. 15 may be an electric field formed by a high frequency of 20 kHz or more, or an electric field generated by a low frequency of 1 kHz or less. When a high frequency is applied, the electric field may penetrate the cells and cause perforations in the cell membranes, and when a low frequency is applied, the electric field does not penetrate the cells, but while flowing along the cell membranes, may cause perforations in the cell membranes. Depending on a frequency range, there is a difference in whether the electric field may be applied to penetrate the cells or to flow along the epidermis, but the common characteristic is that they further cause perforations of the cell membranes by the electric field after causing perforations of the cell membranes by microgravity.

According to the embodiment of “enhanced transfection”, by applying the electric field in the zero gravity or microgravity section of the internal space 124, a greater stress may be induced on the cell membranes of the target cells C, thereby promoting a cell deformation state. Thereby, it is possible to greatly improve the degree to which specific substances D are transfected through the cell membranes. In this case, since perforations of the cell membranes have been already caused due to the change in gravity, even when a low-intensity electric field is applied compared to the electric field applied in the existing electroporation, the degree of material penetration may be greatly improved. Therefore, it is possible to apply the electric field within a range of a critical value at which the target cells C are destroyed or below, and the present invention has the advantage of greatly increasing the cell membrane permeability while avoiding the problem of cell destruction that has been previously pointed out.

Meanwhile, it is also possible to implement electro-gravitoporation of still another embodiment, which combines the embodiments described in FIGS. 14 and 15. As one example, a combination of the electro-gravitoporation of FIGS. 14 and 15 is also possible.

Still another embodiment may be performed by combining the “preconditioning” embodiment in which the cell deformation state is induced according to pretreatment of the target cells C by applying the electric field in the acceleration section of the internal space 124 and the “enhanced transfection” embodiment in which a greater stress is induced on the cell membranes of the target cells C by applying the electric field in the zero gravity or microgravity section of the internal space 124, thereby promoting the cell deformation state. In this way, the present invention has the advantage of significantly increasing the cell membrane permeability because the above embodiment is a method in which gravitoporation is performed after the electric field is applied to the target cells C and then an additional electric field is applied.

As described above, the effective gravity and electric field control device 1000 of the present invention allows the gravitoporation technology and the electro-gravitoporation technology to be performed in combination in various stages, and thus has the advantage of increasing cell membrane permeability of target cells C and processing a large number of cells.

In the above, the embodiments of the present invention have been described with reference to the accompanying drawings, and those of ordinary skill in the art to which this subject matter pertains could understand that the additional or alternative embodiments may be embodied in other specific forms without departing from the technical spirit or essential features of the present invention. Therefore, it is to be appreciated that the embodiments described above are intended to be illustrative in all respects and not restrictive.

What is claimed is:

1. An effective gravity and electric field control device that implements a zero gravity, microgravity, low gravity, or weighted gravity state in a target cell, the effective gravity and electric field control device comprising:
an external module in which a movement space of an internal module is provided and of which at least one physical quantity related to movement is controlled;
the internal module moving independently from the external module within the movement space by an external force transmitted through the external module;
a position detection sensor configured to detect a position of the internal module within the movement space;
a controller configured to generate a control signal for controlling at least one physical quantity related to the movement of the external module based on the position of the internal module detected by the position detection sensor; and
an actuator configured to move the external module so that the external module has the at least one physical quantity according to the control signal,
wherein the internal module includes an internal space where the target cell and a specific substance to be transfected into the target cell are loaded in a suspension state.

2. The effective gravity and electric field control device of claim 1, wherein
the controller is configured to:
generate a first control signal for controlling the actuator so that an external force is applied to the internal module through the external module in a first section; and
generate a second control signal for controlling the actuator by referring to the position of the internal module after the external force applied to the internal module is released in a second section after the first section.

3. The effective gravity and electric field control device of claim 2, wherein the first section is an acceleration section and the second section is a microgravity section.

4. The effective gravity and electric field control device of claim 1, further comprising an electrode unit configured to apply current to the internal space,
wherein the electrode unit includes a plurality of electrodes installed along a wall of the internal module.

5. The effective gravity and electric field control device of claim 4, wherein the electrode unit further includes:
a current source configured to supply current to the plurality of electrodes; and
a multiplexer configured to selectively distribute signals applied from the current source to the plurality of electrodes, and
the current source and the multiplexer form an electric field and a current flow in various directions in the internal space through the plurality of electrodes.

6. The effective gravity and electric field control device of claim 4, wherein the controller is configured to generate an electric signal for forming an electric field in the internal space through the electrode unit in the first section or the second section.

7. The effective gravity and electric field control device of claim 6, wherein the controller is configured to form an electric field in the internal space through the electrode unit by generating the electric signal, in the first section in which the external module and the internal module move by the first control signal.

8. The effective gravity and electric field control device of claim 6, wherein the controller is configured to form an electric field in the internal space through the electrode unit by generating the electric signal, in the second section in which the external module and the internal module move by the second control signal.

9. The effective gravity and electric field control device of claim 4, wherein the internal module includes a gravity measuring unit configured to measure an effective gravity applied to the internal module.

10. The effective gravity and electric field control device of claim 9, wherein the controller is configured to generate an electric signal to be applied to the internal module based on the effective gravity of the internal module measured by the gravity measuring unit.

11. The effective gravity and electric field control device of claim 10, wherein the controller is configured to Perform control to form an electric field and current distribution from the electrode unit to the internal space when the internal module is in a weighted gravity state based on the signal measured from the gravity measuring unit.

12. The effective gravity and electric field control device of claim 10, wherein the controller is configured to perform control to form an electric field and current distribution from the electrode unit to the internal space when the internal module is in a microgravity or zero gravity state based on the signal measured from the gravity measuring unit.
